(19) United States
(12) Patent Application Publication
(10) Pub. No.: US 2008/0144770 A1
(43) Pub. Date: Jun. 19, 2008

(54) Radiation Treatment System Having a Function of Compensating Displacement According to Breathing and Method for Controlling the System

(76) Inventors: Suk Lee, Seoul (KR); Chul Yong Kim, Seoul (KR); Dae Sik Yang, Seoul (KR); Myung Sun Choi, Seoul (KR)

Correspondence Address: CHRISTIE, PARKER & HALE, LLP, PO BOX 7068, PASADENA, CA, 91109-7068, US

(21) Appl. No.: 11/792,196

(22) PCT Filed: Dec. 6, 2005
(86) PCT No.: PCT/KR2005/004153

§ 371 Date: Jun. 1, 2007

(30) Foreign Application Priority Data
Dec. 9, 2004 (KR) 10-2004-0103433
Dec. 5, 2005 (KR) 10-2005-0117757

(51) Int. Cl. A61N 5/10

(52) U.S. Cl. 378/65

(57) ABSTRACT

A radiotherapy system capable of compensating for displacement according to the breathing of a patient, and a method for controlling the same are disclosed. The method includes the steps of: a) irradiating an affected part of the patient who lies on a moving phantom which compensates for displacement of the irradiated part according to breathing of the patient; b) measuring a distance from the irradiated part of the patient to each of ultrasonic sensors using ultrasound; c) extracting displacement of the irradiated part of the patient on the basis of the measured distance; d) generating displacement-compensation information for compensating for the extracted displacement of the irradiated part; and e) moving the moving phantom on the basis of the displacement-compensation information, and compensating for the displacement of the irradiated part. The radiotherapy system acquires the displacement according to the breathing of a patient using an ultrasonic sensor, and adjusts the location of a moving phantom (i.e., a patient bed) using an inverse value of the acquired displacement, such that it recognizes displacement caused by the natural breathing of the patient, reduces a target volume, and ensures patient safety.

### TECHNICAL FIELD

The present invention relates to a radiation therapy system (also called a radiotherapy system) capable of compensating for displacement according to the breathing of a patient, and a method for controlling the same.

### BACKGROUND ART

In a radiotherapy process, precise tumor localization with inter-fraction and intra-fraction motion is a very important factor in improving the precision of radiotherapy.

Precision in tumor localization is affected by correspondence between an anatomic target and a surface marking, an inter-fractional variation of internal organs, and an intra-fractional motion of internal organs, etc. In this case, the intra-fractional motion in internal organs is caused by the breathing of a patient.

Particularly, if the radiotherapy is applied to a tumor located at the upper abdomen of a patient, such as the lungs and the liver, there arises tumor displacement caused by the breathing of the patient (i.e., a respiratory tumor displacement) during the radiotherapy, such that a target volume increases, thereby increasing radiation toxicity and dose in normal tissues.

Furthermore, a current attitude of the patient is unavoidably changed to another attitude due to the breathing of the patient (i.e., a patient's respiratory movement) during the radiotherapy, such that it is difficult to perform precise tumor localization. Thus, there is a need to develop a system capable of accurately defining a lesion and intensively applying an appropriate dose of radiation to the target.

Radiotherapy based on quantified respiration data, X-ray image-based radiotherapy, artificial tracer-guided radiotherapy, and skin marker-guided radiotherapy, etc. are known as breathing compensation-type radiotherapy techniques capable of compensating for an internal organ motion according to the breathing of patients.

However, these techniques separately require expensive therapeutic equipment and can be employed only with specific radiotherapy equipment, such that they restrict general application of these techniques.

Further, conventional breathing compensation-type radiotherapy systems adopt an on/off switch mode for radiation, which shortens the durability of the systems, such that it is inappropriate to use them in domestic circumstances.

In particular, radiotherapy based on quantified respiration data involves some risk such as artificial respiration control. In view of the foregoing, there is a need to develop a novel radiotherapy system capable of rendering effective radiation treatment in consideration of normal breathing of patients without involving any risk.

### DISCLOSURE

### Technical Problem

Therefore, the present invention has been made in view of the above problems, and it is an object of the present invention to provide a radiotherapy system capable of compensating for displacement according to the breathing of a patient, and a method for controlling the same.

### Technical Solution

In accordance with one aspect of the present invention, the above and other objects can be accomplished by the provision of a method for controlling a radiotherapy system equipped with a displacement compensation function according to breathing of a patient, the method comprising the steps of: a) irradiating an affected part of the patient who lies on a moving phantom (100) which compensates for displacement of the irradiated part according to breathing of the patient; b) measuring a distance from the irradiated part of the patient to each of ultrasonic sensors (101) using ultrasound; c) extracting displacement of the irradiated part of the patient on the basis of the measured distance; d) generating displacement-compensation information for compensating for the extracted displacement of the irradiated part; and e) moving the moving phantom on the basis of the displacement-compensation information, and compensating for the displacement of the irradiated part.

Preferably, the step b) includes the steps of: transmitting the ultrasound to a skin (i.e., epidermis) corresponding to the irradiated part of the patient several times within a predetermined period of time; receiving the ultrasound reflected from the skin; and measuring a distance from the skin to each of the ultrasonic sensors.

In accordance with another aspect of the present invention, there is provided a radiotherapy system equipped with a displacement compensation function according to breathing of a patient comprising: a displacement compensator (103) for compensating for a patient location such that a skin location corresponding to a predetermined affected part of the patient is not changed according to breathing of the patient; a moving phantom unit (100) equipped with a controller (102) which includes a data collector (1021) for measuring a distance from the skin location to the ultrasonic sensor using an ultrasonic sensor (101), and a drive (1022) for receiving displacement-compensation information compensating for the skin displacement corresponding to the measured distance, and controlling the displacement compensator; a displacement analysis unit (201) for receiving information associated with the measured distance from the data collector (1021), and extracting displacement of the skin on the basis of the received information; a displacement-information provider (200) for receiving the extracted displacement information from the displacement analysis unit (201), generating displacement-compensation information of a direction opposite to the displacement, and providing the drive (1022) with the generated displacement-compensation information; and a radiation exposure unit (900) for irradiating the affected part of the patient.

Preferably, the ultrasonic sensor (101) is composed of a plurality of ultrasonic sensors such that a distance from the patient skin to each of the ultrasonic sensors can be measured in X-, Y-, and Z-axes directions, and the displacement compensator (103) compensates for the patient location in X-, Y-, and Z-axes directions.

Preferably, the moving phantom (100) includes: a first moving unit (1031) including a bed unit (10312) on which the patient can lie, and a first moving means (10311) movable in an X-, or Y-axis direction according to a control signal of the controller (102); a second moving unit (1032) for supporting the first moving unit (1031) from below, and including a second moving means movable in an X-, or Y-axis direction perpendicular to a moving direction of the first moving unit (1031) according to a control signal of the controller (102); a third moving unit (1033) for supporting the second moving unit (1032) from below, and including a third moving means (10331) movable in a Z-axis direction indicative of a perpendicular direction according to a control signal of the controller (102); and a bottom unit (104) for supporting the third moving unit (1033) from below, and including a sensor support (1011) for supporting the ultrasonic sensors (101) which measure the distance from the patient skin to each of the ultrasonic sensors in X-, Y-, and Z-axes directions of two sides adjacent to each other.

Preferably, the first, second, or third moving unit (10311, 10321, or 10331) corresponds to any one of a rack/pinion, a bevel gear, a worm gear, and a piston.

### Advantageous Effects

The radiotherapy system and method according to the present invention adjusts a moving phantom (i.e., a patient's bed) using an inverse value of respiratory patient movement acquired by an ultrasonic sensor, such that it recognizes displacement caused by the natural breathing of the patient, thereby reducing a target volume and ensuring patient safety.

### DESCRIPTION OF DRAWINGS

The above and other objects, features and other advantages of the present invention will be more clearly understood from the following detailed description taken in conjunction with the accompanying drawings, in which:

FIG. 1 is a view illustrating a radiotherapy system capable of compensating for displacement according to the breathing of a patient in accordance with the present invention;

FIG. 2 is a block diagram illustrating a radiotherapy system capable of compensating for displacement according to breathing of a patient in accordance with a preferred embodiment of the present invention;

FIG. 3 is a flow chart illustrating a method for controlling the radiotherapy system capable of compensating for displacement according to the breathing of a patient in accordance with a preferred embodiment of the present invention;

FIGS. 4A˜4B are a perspective view and a side view of a moving phantom of the radiotherapy system capable of compensating for displacement according to the breathing of a patient in accordance with the present invention, respectively;

FIG. 5 is a graph illustrating a comparison between displacement information acquired by the radiotherapy system equipped with a displacement compensation function according to the breathing of a patient and displacement-compensated information in accordance with the present invention;

FIG. 6 is a three-dimensional graph illustrating a comparison between displacement information acquired by the radiotherapy system equipped with a displacement compensation function according to the breathing of a patient and displacement-compensated information in accordance with the present invention;

FIG. 7 is a time-variant displacement graph illustrating displacement information acquired by the radiotherapy system equipped with a displacement compensation function according to the breathing of a patient and displacement-compensated information in accordance with the present invention; and

FIG. 8 is a time-variant table illustrating displacement information acquired by the radiotherapy system equipped with a displacement compensation function according to the breathing of a patient and displacement-compensated information in accordance with the present invention.

### BEST MODEL

A radiotherapy system capable of compensating for displacement according to the breathing of a patient and a method of controlling the radiotherapy system according to the present invention will now be described more fully with reference to the accompanying drawings, in which exemplary embodiments of the invention are shown.

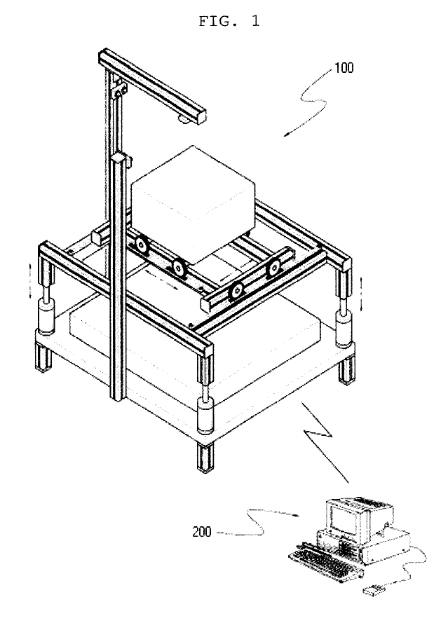

FIG. 1 is a view illustrating a radiotherapy system capable of compensating for displacement according to the breathing of a patient in accordance with the present invention.

Referring to FIG. 1, the radiotherapy system capable of compensating for displacement according to the breathing of a patient in accordance with a preferred embodiment of the present invention includes a moving phantom unit 100 and a compensation-information provider 200.

The moving phantom unit 100 measures displacement of the skin corresponding to a patient's treatment part, and moves in the direction of offsetting the displacement of the patient.

The compensation-information provider 200 receives the measured skin-displacement information, generates displacement-compensated information capable of changing the patient location on the basis of the received skin-displacement information, and transmits the generated displacement-compensated information to the moving phantom unit 100.

The radiotherapy system according to the present invention includes a radiation exposure unit 900 (not shown in FIG. 1) for irradiating the affected part of a patient (i.e., a treatment target part). The radiation exposure unit 900 is equal to that of the conventional radiotherapy system, so that a detailed description thereof will herein be omitted for the convenience of description.

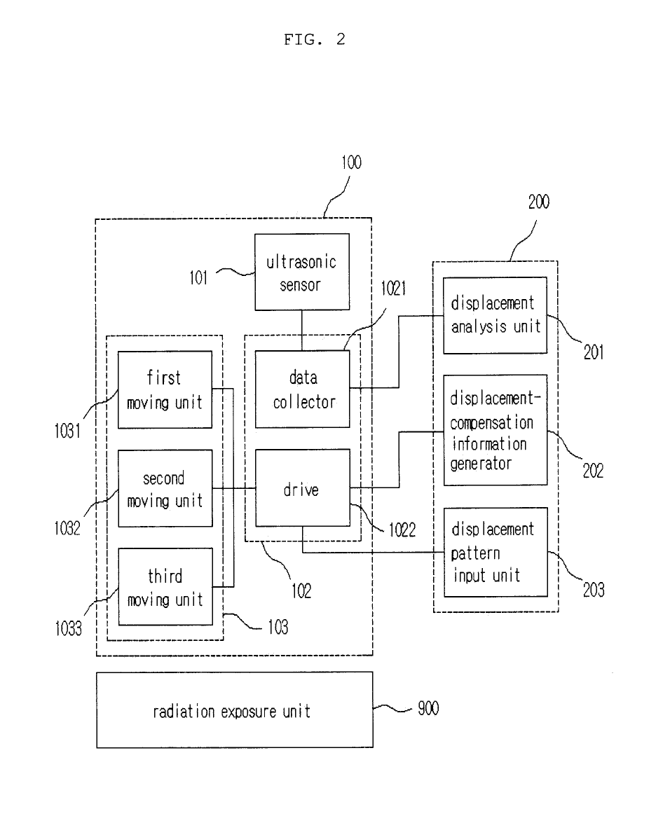

FIG. 2 is a block diagram illustrating a radiotherapy system capable of compensating for displacement according to breathing of a patient in accordance with a preferred embodiment of the present invention.

Referring to FIG. 2, the moving phantom unit 100 includes an ultrasonic sensor 101, a controller 102, and a displacement compensator 103. The controller 102 includes a data collector 1021 and a drive 1022. The displacement compensator 103 includes first and second moving units 1031 and 1032. If required, the displacement compensator 103 may further include a third moving unit 1033.

The compensation-information provider 200 according to the present invention includes a displacement analysis unit 201 and a displacement-compensation information generator 202. If required, the compensation-information provider 200 may further include a displacement pattern input unit 203.

The ultrasonic sensor 101 measures a distance from the skin corresponding to the patient's affected part to be irradiated to the ultrasonic sensor itself using ultrasound. If required, a plurality of ultrasonic sensors 101 may be arranged in the directions of X-, Y-, and Z-axes.

The data collector 1021 controls the above-mentioned ultrasonic sensor 101 to measure the distance from the above-mentioned skin to the ultrasonic sensor 101. If required, the data collector 1021 measures the distance from the ultrasonic sensor 101 to the above-mentioned skin several times within a predetermined period of time (e.g., 10 ms), such that it may determine the final distance from the ultrasonic sensor 101 to the above-mentioned skin on the basis of the measured result.

The drive 1022 receives displacement-compensation information from either the displacement-compensation information generator 202 or the displacement pattern input unit 203, and controls the displacement compensator 103 to move the patient location in the direction of X-, Y-, or Z-axis.

The displacement compensator 103 changes the patient location according to a control signal of the drive 1022.

The first and second moving units 1031 and 1032 of the displacement compensator 103 move the patient location in the direction of X- or Y-axis. The third moving unit 1033 moves the patient location in the direction of Z-axis.

The displacement analysis unit 201 receives distance information measured by the data collector 1021, and extracts individual displacements measured in X-, Y-, and Z-axes directions of the above skin on the basis of the received distance information.

The displacement-compensation information generator 202 receives the skin-displacement information in the directions of X-, Y-, and Z-axes from the displacement analysis unit 201, and generates displacement-compensation information capable of moving the above patient in the directions opposite to the above-mentioned X-, Y-, and Z-axes directions.

If the patient location is adjusted or is required to move according to a predetermined pattern, the displacement pattern input unit 203 receives displacement-compensation information for moving the patient location from an external part, and provides the drive 1022 with the received displacement-compensation information.

The radiation exposure unit 900 irradiates the affected part of the patient.

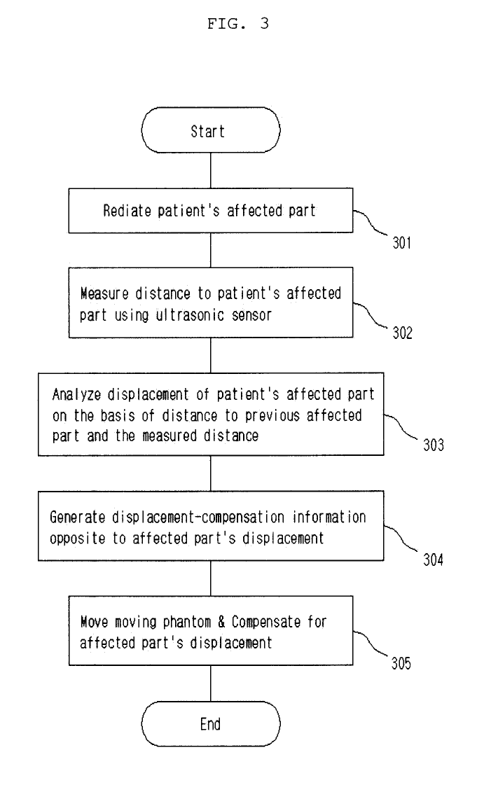

FIG. 3 is a flow chart illustrating a method for controlling the radiotherapy system capable of compensating for displacement according to the breathing of a patient in accordance with a preferred embodiment of the present invention.

Referring to FIG. 3, the radiotherapy system measures a distance from the patient's affected part (i.e., the affected skin) to the ultrasonic sensor using the ultrasonic sensor at step 302. The radiotherapy system analyzes the displacement of the patient's affected part in the directions of X-, Y-, or Z-axis using previously-measured distance information and currently-measured distance information at step 303.

Thereafter, the radiotherapy system generates displacement-compensation information capable of moving the patient location in the direction opposite to the displacement of the above affected part at step 304, and moves the moving phantom on which the patient lies on the basis of the generated displacement-compensation information, such that it compensates for the displacement of the patient's affected part at step 305.

In this case, the radiotherapy system performs the step 301 of irradiating the patient's affected part during the execution of the above steps 302˜305.

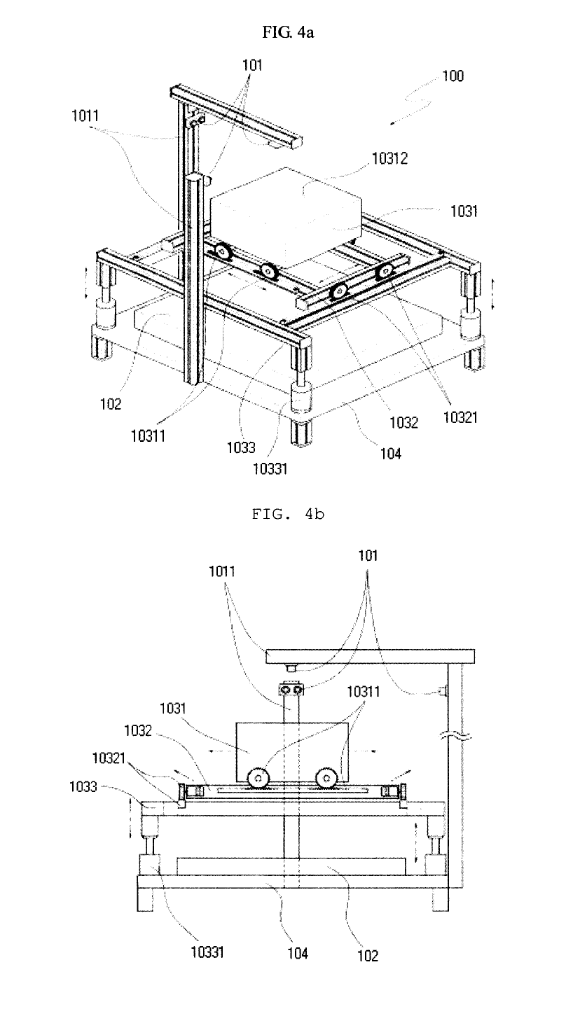

FIGS. 4A˜4B are a perspective view and a side view of a moving phantom of the radiotherapy system capable of compensating for displacement according to the breathing of a patient in accordance with the present invention, respectively. The moving phantom according to the present invention will hereinafter be described with reference to FIGS. 4A˜4B.

Referring to FIGS. 4A˜4B, the first moving unit 1031 includes a bed 10312 on which the patient can lie, and also includes a first moving means 10311 movable in the direction of X- or Y-axis according to a control signal of the controller 102.

FIGS. 4A˜4B show the example of the first moving means 10311 implemented with a rack and a pinion. In this case, the rack is configured in the form of a wheel which can move the first moving unit 1031 in the direction of X- or Y-axis along the pinion formed on the upper surface of the second moving unit. The axis of the above-mentioned rack is connected to a servo motor driven by power- and control- signals received from the controller 102. The servo motor is contained in the first moving unit 1031.

The second moving unit 1032 supports the first moving unit 1031 from below. The second moving unit 1032 includes a second moving means 10321. The second moving means 10321 is movable in a horizontal direction perpendicular to the moving direction of the first moving unit 1031 according to a control signal of the controller 102. For example, if the first moving unit 1031 moves in the X-axis direction, the second moving unit 1032 is movable in the Y-axis direction.

FIGS. 4A˜4B show the example of the second moving means 10321 implemented with a rack and a pinion. In this case, the rack is configured in the form of a wheel which can move the second moving unit 1032 in the direction of X- or Y-axis along the pinion formed on the upper surface of either a third moving means or the bottom 104. The axis of the above-mentioned rack is connected to a servo motor driven by power- and control-signals received from the controller 102. The servo motor is contained in the second moving unit 1031.

The third moving unit 1033 supports the second moving unit 1032 from below. The third moving unit 1033 includes a third moving means 10331. The third moving means 10331 is movable in a vertical direction (i.e., Z-axis direction) according to a control signal of the controller 102. FIGS. 4A-4B show the example of the third moving unit 1033 implemented with a piston.

The first moving means 10311, the second moving means 10321, or the third moving means 10331 may be implemented with a bevel gear, a rack/pinion, a worm gear, or a piston, etc.

The bottom 104 supports the second or third moving unit 1032 or 1033 from below. A sensor support 1011 for supporting the ultrasonic sensor 101 is located at both sides of two directions (i.e., X- and Y-axes).

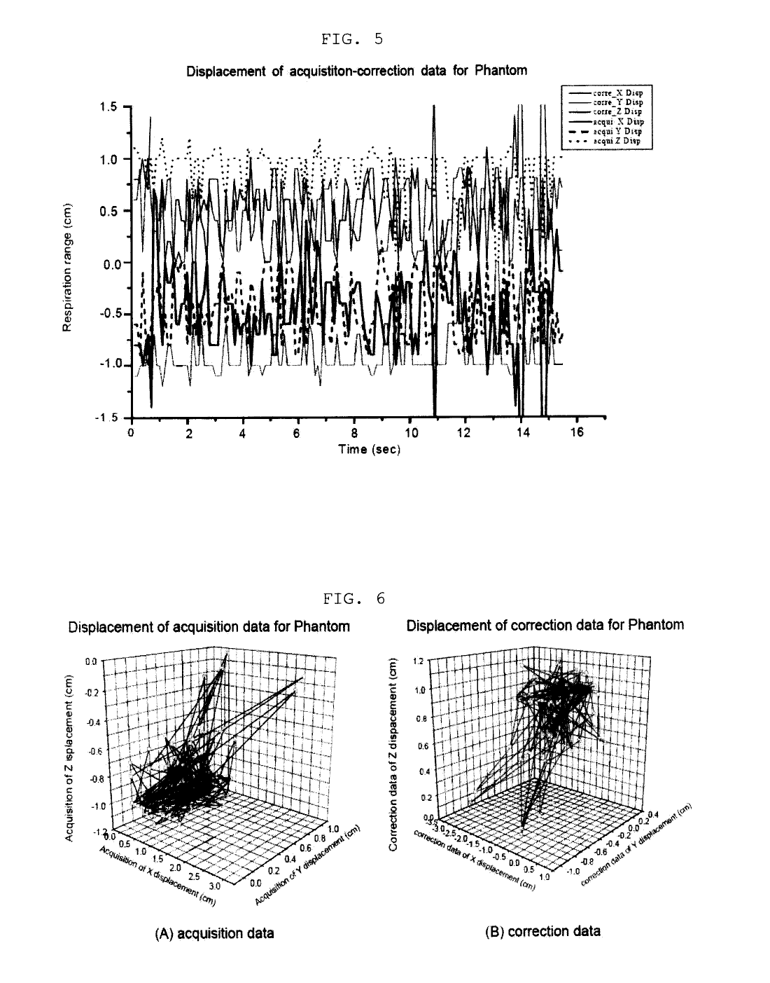

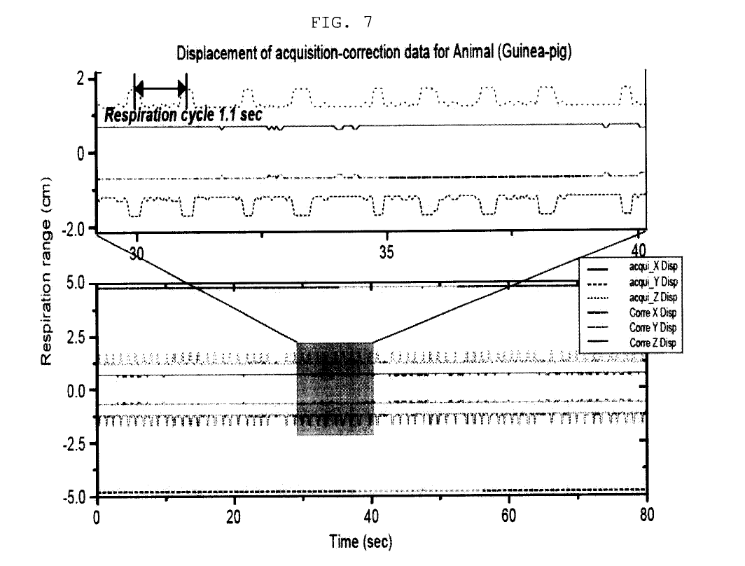

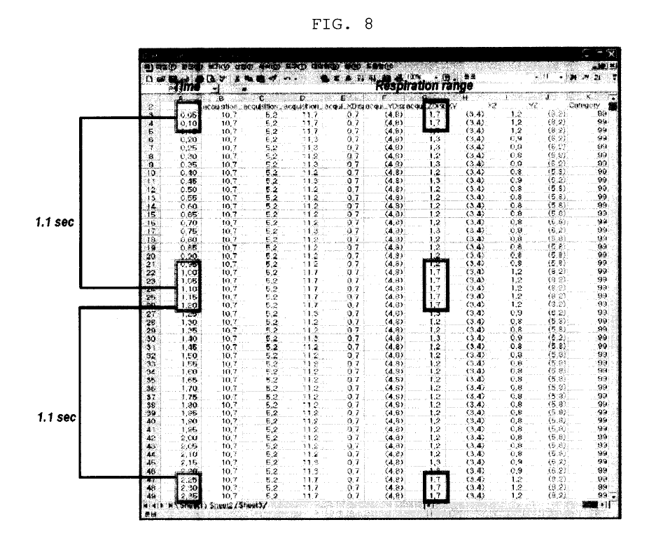

FIG. 5 is a graph illustrating a comparison between displacement information acquired by the radiotherapy system equipped with a displacement compensation function according to the breathing of a patient and displacement-compensated information in accordance with the present invention. FIG. 6 is a three-dimensional graph illustrating a comparison between displacement information acquired by the radiotherapy system equipped with a displacement compensation function according to the breathing of a patient and displacement-compensated information in accordance with the present invention. FIG. 7 is a time-variant displacement graph illustrating displacement information acquired by the radiotherapy system equipped with a displacement compensation function according to the breathing of a patient and displacement-compensated information in accordance with the present invention. FIG. 8 is a time-variant table illustrating displacement information acquired by the radiotherapy system equipped with a displacement compensation function according to the breathing of a patient and displacement-compensated information in accordance with the present invention.

The experiments in FIGS. 5 to 8 are required to acquire/compensate for displacement of the skin of a guinea pig having a weight of about 500g according to the breathing of the guinea pig. In more detail, in order to acquire skin-displacement data of the guinea pig according to the periodic breathing of the guinea pig, the guinea pig is locally anesthetized, and the displacement data is acquired for 80 seconds.

According to the results of the above-mentioned experiments, a delay time between acquisition data and corresponding correction data is equal to the sum of ultrasound emission-reception times of the ultrasonic sensor and a displacement-compensation information generation time, and is about 2.34×10⁻⁴ seconds.

Referring to FIG. 5, if the precision between the displacement-measurement data and displacement-compensation data is compared with X-, Y-, and Z-axes variations in time, it can be recognized that acquisition data and correction data in individual directions are mapped in a one-to-one manner.

The graph of FIG. 6 shows the acquisition-correction data in a three-dimensional space, such that a user can visually evaluate the displacements in individual directions using the graph of FIG. 6.

FIG. 7 is a time-variant displacement graph illustrating a variation in acquisition data of the skin-displacement caused by the breathing of the guinea pig and a variation in correction data acquired by inverse values of lateral acquisition data of the above displacement. FIG. 8 is a time-variant table illustrating the above-mentioned variations of the acquisition data and the correction data.

As shown in FIGS. 7˜8, there is little displacement in horizontal and vertical directions in the above-mentioned acquisition data, the displacement in front and rear directions corresponds to a maximum of 5 mm, and a respiration period corresponds to 1.1 seconds.

If the precision between the acquisition data and the correction data is compared with a distance in time, it can be recognized that the acquisition data and the correction data are mapped in a one-to-one manner with the precision of±1% in individual directions.

In the meantime, the methods according to the present invention can be configured in the form of a computer program executable by a computer. The above-mentioned computer program can be recorded in a computer-readable recording medium (e.g., a Compact Disc, a Hard Disc Drive, a floppy disc, and other memories, etc.).

### INDUSTRIAL APPLICABILITY

The radiotherapy system and method according to the present invention acquires the displacement according to the breathing of a patient using an ultrasonic sensor, and adjusts the location of a moving phantom (i.e., a patient bed) using an inverse value of the acquired displacement. Therefore, the radiotherapy system and method recognizes displacement caused by the natural breathing of the patient, reduces a target volume, and ensures patient safety.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method for controlling a radiotherapy system equipped with a displacement compensation function according to breathing of a patient, the method comprising:
a) irradiating an affected part of the patient on a moving phantom unit adapted to compensate for a displacement of the irradiated part according to breathing of the patient;
b) measuring a distance from the irradiated part of the patient to each of ultrasonic sensors using ultrasound;
c) extracting a displacement of the irradiated part of the patient in accordance with the measured distance;
d) generating displacement-compensation information to compensate for the extracted displacement of the irradiated part; and
e) moving the moving phantom unit in a direction opposite to the displacement in accordance with the displacement-compensation information during a respiration period, and compensating for the displacement of the irradiated part.

2. The method according to claim 1, wherein the the measuring the distance b) comprises:
transmitting the ultrasound to a skin of the patient corresponding to the irradiated part of the patient for several times within a period of time;
receiving the ultrasound reflected from the skin; and
measuring a distance from the skin to each of the ultrasonic sensors.

3. A computer-readable recording medium comprising a computer program for causing a computer to execute a method comprising:
irradiating an affected part of the patient on a moving phantom unit adapted to compensate for a displacement of the irradiated part due to breathing of the patient;
measuring a distance from the irradiated part of the patient to each of ultrasonic sensors using ultrasound;
extracting a displacement of the irradiated part of the patient in accordance with the measured distance;
generating displacement-compensation information to compensate for the extracted displacement of the irradiated part; and
moving the moving phantom unit in a direction opposite to the displacement in accordance with the displacement-compensation information during a respiration period, and compensating for the displacement of the irradiated part.

4. A radiotherapy system equipped with a displacement compensation function according to breathing of a patient comprising:
a displacement compensator for compensating for a patient location in a direction opposite to a displacement of an affected part of the patient such that a skin location corresponding to the affected part of the patient is not substantially changed due to the breathing of the patient during a respiration period;
a moving phantom unit equipped with a controller, the controller comprising:
a data collector for measuring a distance from the skin location to the ultrasonic sensor using an ultrasonic sensor, and
a drive for receiving displacement-compensation information to compensate for the displacement corresponding to the measured distance and to control the displacement compensator;
a displacement analysis unit for receiving information associated with the measured distance from the data collector, and extracting a displacement of the skin in accordance with the received information;
a displacement-information provider for receiving the extracted displacement information from the displacement analysis unit, generating the displacement-compensation information in the direction opposite to the displacement, and providing the drive with the generated displacement-compensation information; and
a radiation exposure unit for irradiating the affected part of the patient.

5. The radiotherapy system according to claim 4, wherein:
the ultrasonic sensor comprises a plurality of ultrasonic sensors such that a distance from the patient skin to each of the ultrasonic sensors can be measured in first, second, and third directions, and
the displacement compensator is adapted to compensate for the patient location in the first, second, and third directions.

6. The radiotherapy system according to claim 4, wherein the moving phantom comprises:
a first moving unit including a bed unit on which the patient can lie, and first moving means for moving in the first direction according to a control signal of the controller;
a second moving unit for supporting the first moving unit from below, and including second moving means for moving in the second direction perpendicular to the first direction of the first moving unit according to a control signal of the controller; and
a bottom unit for supporting the second moving unit from below, and including a sensor support for supporting the ultrasonic sensors adapted to measure the distance from the patient skin to each of the ultrasonic sensors in the first, second, and third directions and at two sides of the bottom unit adjacent to each other.

7. The radiotherapy system according to claim 6, further comprising:
a third moving unit for supporting the second moving unit from below, and including third moving means for moving in the third direction perpendicular to the first and second directions according to a control signal of the controller, and
the bottom part being configured to support the third moving unit from below.

8. The radiotherapy system according to claim 7, wherein each of the first, second, or third moving units comprises a least one of a rack/pinion, a bevel gear, a worm gear, or a piston.

9. The method according to claim 2, wherein the transmitting the ultrasound to the skin of the patient comprises transmitting the ultrasound to an epidermis of the patient corresponding to the irradiated part of the patient for several times within the period of time.

10. The method of claim 3, wherein the measuring the distance comprises:
transmitting the ultrasound to a skin of the patient corresponding to the irradiated part of the patient for several times within a period of time;
receiving the ultrasound reflected from the skin; and
measuring a distance from the skin to each of the ultrasonic sensors.

11. The method of claim 10, wherein the transmitting the ultrasound to the skin of the patient comprises transmitting the ultrasound to an epidermis of the patient corresponding to the irradiated part of the patient for several times within the period of time.

12. The radiotherapy system according to claim 6, wherein the first direction is an X-axis direction and the second direction is a Y-axis direction.

13. The radiotherapy system according to claim 6, wherein the first direction is a Y-axis direction and the second direction is an X-axis direction.

14. The radiotherapy system according to claim 7, wherein the first direction is an X-axis direction, the second direction is a Y-axis direction, and the third direction is a Z-axis direction.

15. The radiotherapy system according to claim 7, wherein the first direction is a Y-axis direction, the second direction is an X-axis direction, and the third direction is a Z-axis direction.
